(19) United States
(12) Patent Application Publication
(10) Pub. No.: US 2026/0198953 A1
(43) Pub. Date: Jul. 16, 2026

(54) MEDICAL DEVICE

(71) Applicant: Arbutus Medical Inc., Vancouver (CA)

(72) Inventors: Michael Cancilla, Vancouver (CA); Radu Postole, Abottsford (CA)

(73) Assignee: Arbutus Medical Inc., Vancouver (CA)

(21) Appl. No.: 19/016,611

(22) Filed: Jan. 10, 2025

(51) Int. Cl. A61B 17/32; A61B 17/00

(52) U.S. Cl. CPC A61B 17/32; A61B 2017/00407; A61B 2017/00477; A61B 2017/00526; A61B 2017/00946

(57) ABSTRACT

The present disclosure is directed to a medical device. The medical device can include a first molded arm having a first aperture and a first flexor monolithically coupled with the first molded arm. The medical device can include a second molded arm coupled with the first molded arm, the second molded arm having a second aperture and a second flexor monolithically coupled with the second molded arm. The medical device can include a first tooth coupled with the first molded arm at the first aperture. The medical device can include a second tooth coupled with the second molded arm at the second aperture. A least one of the first tooth or the second tooth can have a cutting edge in a range of 0.10 mm to 0.17 mm.

### BACKGROUND

Various medical devices can be used to support several bones, muscles, ligaments, or tendons in a body.

### SUMMARY

At least one aspect is directed to a rongeur medical device. The rongeur medical device can include a first molded arm having a first aperture and a first flexor monolithically coupled with the first molded arm. The rongeur medical device can include a second molded arm coupled with the first molded arm, the second molded arm having a second aperture and a second flexor monolithically coupled with the second molded arm. The rongeur medical device can include a first tooth coupled with the first molded arm at the first aperture. The rongeur medical device can include a second tooth coupled with the second molded arm at the second aperture. A least one of the first tooth or the second tooth can have a cutting edge in a range of 0.10 mm to 0.17 mm.

At least one aspect is directed to a method. The method can include forming, via molding, a first arm, the first arm including a first flexor and a first aperture. The method can include forming, via molding, a second arm, the second arm including a second flexor and a second aperture. The method can include coupling the first arm with the second arm and the first flexor with the second flexor. The method can include forming, via forging, a first tooth. The method can include forming, via forging, a second tooth. The method can include coupling the first tooth with the first arm at the first aperture. The method can include coupling the second tooth with the second arm at the second aperture.

At least one aspect is directed to a method. The method can include providing a rongeur medical device. The rongeur medical device can include a first molded arm having a first aperture and a first flexor monolithically coupled with the first molded arm. The rongeur medical device can include a second molded arm coupled with the first molded arm, the second molded arm having a second aperture and a second flexor monolithically coupled with the second molded arm. The rongeur medical device can include a first tooth coupled with the first molded arm at the first aperture. The rongeur medical device can include a second tooth coupled with the second molded arm at the second aperture. A least one of the first tooth or the second tooth can have a cutting edge in a range of 0.10 mm to 0.17 mm.

At least one aspect is directed to a freer elevator medical device. The freer elevator medical device can include a first end, a second end, and a body extending between the first end and the second end. The freer elevator medical device can include a bulge coupled with the body and disposed between the first end and the second end, a width of the bulge being greater than a width of the body. The first end can include a wedge shape that tapers in height from the body to a first tip. The first tip can be substantially aligned with a center axis of the body. The second end can include a curved shape that tapers in height from the body to a second tip. The second tip can curve away from the center axis of the body to define a radius of curvature in a range of 30 mm to 60 mm. The second tip can have a height at an end most portion of the second tip in a range of 0 mm to 1 mm.

At least one aspect is directed to a method. The method can include providing the freer elevator medical device. The freer elevator medical device can include a first end, a second end, and a body extending between the first end and the second end. The first end can include a wedge shape that tapers in height from the body to a first tip. The first tip can be substantially aligned with a center axis of the body. The second end can include a curved shape that tapers in height from the body to a second tip. The second tip can curve away from the center axis of the body to define a radius of curvature in a range of 30 mm to 60 mm. The method can include manipulating, by the first tip of the freer elevator medical device, periosteum of a digit of a hand or a foot. The method can include elevating, by the second tip of the freer elevator medical device, a nail of the digit.

At least one aspect is directed to a method. The method can include providing a freer elevator medical device. The freer elevator medical device can include a first end, a second end, and a body extending between the first end and the second end. The freer elevator medical device can include a bulge coupled with the body and disposed between the first end and the second end, a width of the bulge being greater than a width of the body. The first end can include a wedge shape that tapers in height from the body to a first tip. The first tip can be substantially aligned with a center axis of the body. The second end can include a curved shape that tapers in height from the body to a second tip. The second tip can curve away from the center axis of the body to define a radius of curvature in a range of 30 mm to 60 mm. The second tip can have a height at an end most portion of the second tip in a range of 0 mm to 1 mm.

These and other aspects and implementations are discussed in detail below. The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. In the drawings:

FIG. 1 is an example side view of a medical device, in accordance with implementations.

FIG. 2 is an example front perspective view of the medical device of FIG. 1, in accordance with implementations.

FIG. 3 is an example rear perspective view the medical device of FIG. 1, in accordance with implementations.

FIG. 4 is an example front view of the medical device of FIG. 1, in accordance with implementations.

FIG. 5 is an example exploded view of the medical device of FIG. 1, in accordance with implementations.

FIG. 6 is an example sectional view of the medical device of FIG. 1, cut along line 6-6 in FIG. 4, in accordance with implementations.

FIG. 7 is another example sectional view of the medical device of FIG. 1, cut along line 7-7 in FIG. 1, in accordance with implementations.

FIG. 8 is an example perspective view of a medical device, in accordance with implementations.

FIG. 9 is another example perspective view of the medical device of FIG. 8, in accordance with implementations.

FIG. 10 is an example side view of the medical device of FIG. 8, in accordance with implementations.

FIG. 11 is an example top view of the medical device of FIG. 8, in accordance with implementations.

FIG. 12 is an example front view of the medical device of FIG. 8, in accordance with implementations.

FIG. 13 is an example rear view of the medical device of FIG. 8, in accordance with implementations.

FIG. 14 is an example illustration of a method of assembling the medical device of FIG. 1, in accordance with implementations.

FIG. 15 is an example illustration of a method, in accordance with implementations.

FIG. 16 is an example illustration of a method of using the medical device of FIG. 8, in accordance with implementations.

FIG. 17 is an example illustration of a method, in accordance with implementations.

### DETAILED DESCRIPTION

Following below are more detailed descriptions of various concepts related to, and implementations of, methods, apparatuses, and systems of medical devices. The various concepts introduced above and discussed in greater detail below may be implemented in any of numerous ways.

The present disclosure relates to a medical device, such as a rongeur medical device. The rongeur medical device can include a handle end having a first molded arm and a second molded arm pivotally coupled together. The first molded arm can include an integrally connected flexor. The second molded arm can include an integrally connected flexor. The flexors can couple with one another to bias the first molded arm away from the second molded arm in at least one position. The rongeur medical device can include a cutting end having a first tooth coupled with the first arm and a second tooth coupled with the second arm. The teeth can include a sharp cutting edge to effectively and efficiently cut away bones, tissues, or other portions of small, delicate body parts, such as digits of the hands or foot (e.g., fingers or toes).

The present disclosure relates to a medical device, such as a freer elevator medical device. The freer elevator medical device can include a body that extends between a first end and a second end. The first end can define a first shape and the second end can define a second, different shape. The first end can include two substantially opposing surfaces that converge to a tip to form a wedge-like shape. The first end can extend substantially straight outward from the body to form the wedge-like shape. This shape facilitates providing a stiff, rigid end that can push back tissue of digits of the hand or feet. The second end can include two curved surfaces that oppose each other and converge to a tip that curves away from a center axis of the body of the device (e.g., to form a hook-like shape). The second end can taper from a proximal portion of the device to the curved tip of the second end such that the tip is thinner than the body of the device. This shape facilitates providing a thin enough tip that can extend under a nail of a digit with a thick enough middle section that can provide enough force to elevate the nail from the nail bed. The medical device can include a bulging section of the body that extends in at least one direction outward beyond another portion of the body to prevent the device from rolling along a surface (e.g., a medical tray).

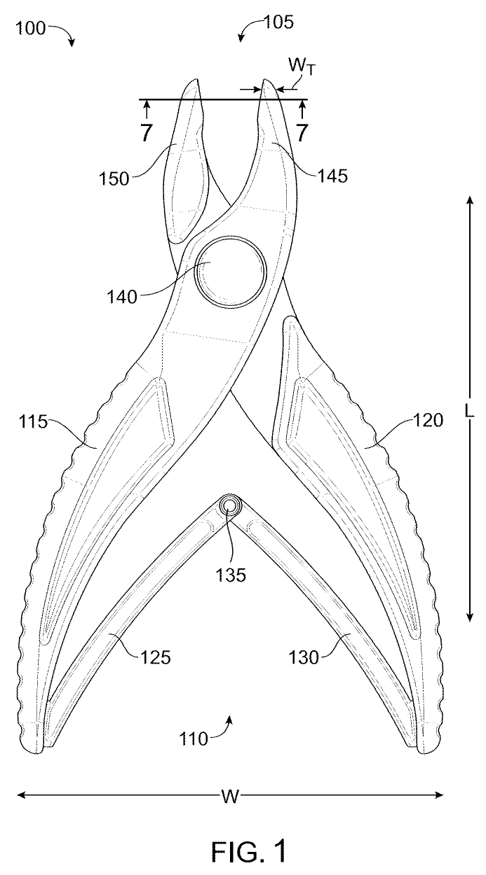

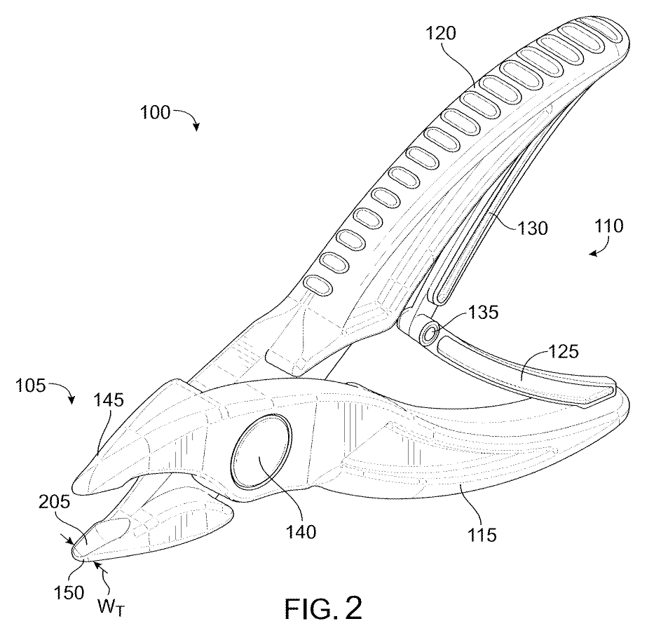

FIG. 1 depicts an example side view of a device (e.g., a medical device) 100 and FIG. 2 depicts an example front perspective view of the medical device 100, according to an example implementation. The medical device 100 can be used in one or more medical settings. For example, the medical device 100 can facilitate healing a patient, the medical device 100 can facilitate one or more medical professionals during operations or other activities, or the medical device 100 can be used in various other applications. For example, the medical device 100 can be used for a patient undergoing various procedures such as, for example, a revision amputator for fingers or toes, finger/nail bed repairs, or various other procedures. As described herein, the medical device 100 can be used to trim or remove small pieces of bones or tissue during a medical procedure. The medical device 100 can be or can include, for example, a rongeur medical device.

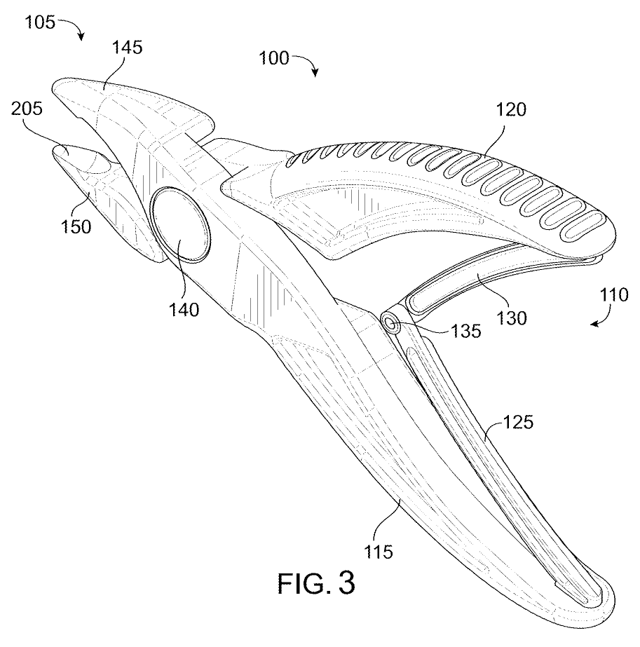

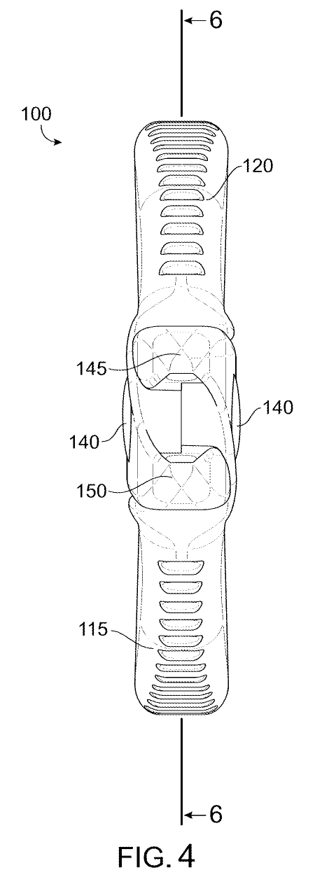

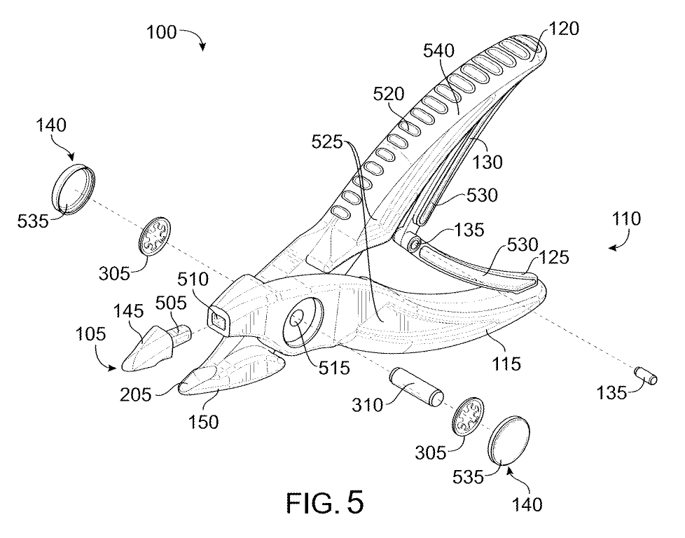

FIG. 3 depicts an example rear perspective view of the medical device 100. FIG. 4 depicts an example front view of the medical device 100. FIG. 5 depicts an example exploded view of the medical device 100. As depicted in at least FIGS. 1-5, the medical device 100 can include a first end 105 and a second end 110. The first end 105 can oppose the second end 110. The first end 105 can be positioned in other various positions relative to the second end 110 (e.g., adjacent to). The first end 105 can be or can include a cutting end and the second end 110 can be or can include a holding end. For example, the first end 105 can include at least one tooth 145. The first end 105 can include a first tooth 145 and a second tooth 150. The first tooth 145 and the second tooth 150 can oppose one another or the first tooth 145 and second tooth 150 can be positioned at various other positions relative to one another. As described herein, the teeth 145, 150 can include a sharp cutting end such that, when the teeth 145, 150 engage with one another, the teeth 145, 150 can cut through one or more materials.

The second end 110 of the medical device 100 can include a handle. For example, the second end 110 can include at least one arm 115. The second end 110 can include a first arm 115 and a second arm 120. As described herein, the first arm 115 and the second arm 120 can be or can include molded arms. The first arm 115 and the second arm 120 can oppose one another or the first arm 115 and second arm 120 can be positioned at various other positions relative to one another. The first arm 115 can extend up to the first tooth 145 and the second arm 120 can extend up to the second tooth 150. For example, the first arm 115 and the second arm 120 can overlap and couple together by one or more pins 140. With this configuration, movement of the first arm 115 and the second arm 120 can cause movement of the first tooth 145 and the second tooth 150 (e.g., as the arms 115, 120 move towards each other, the teeth 145, 150 move towards each other).

The medical device 100 can include one or more biasing mechanisms. For example, the medical device 100 can include a first flexor 125 and a second flexor 130. The first flexor 125 can oppose the second flexor 130. The first flexor 125 can couple with the second flexor 130 by a pin 135 such that the first flexor 125 and the second flexor 130 apply a force at the pin 135 to bias the second end 110 or the medical device 100 to an open position (e.g., the position shown in at least FIG. 1). In other words, the first flexor 125 and the second flexor 130 can bias the first arm 115 and the second arm 120 away from one another (e.g., by applying opposing forces at the pin 135).

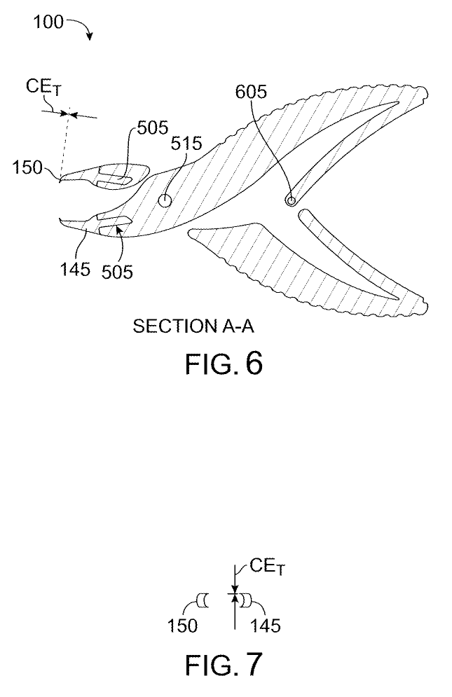

The first arm 115 can couple with the first flexor 125 and the second arm 120 can couple with the second flexor 130. The first arm 115 can monolithically couple with the first flexor 125. For example, the first arm 115 and the first flexor 125 can integrally form together as a unitary or monolithic piece through various processes including, but not limited to, molding. In other words, the first arm 115 and the first flexor 125 can form one monolithic, molded structure. The second arm 120 can monolithically couple with the second flexor 130. For example, the second arm 120 and the second flexor 130 can integrally form together as a unitary or monolithic piece through various processes including, but not limited to, molding. In other words, the second arm 120 and the second flexor 130 can form one, monolithic molded structure. As depicted in at least FIGS. 5 and 6, the first arm 115 and first flexor 125 (and the second arm 120 and the second flexor 130) can be formed via molding with a first hole 515 or aperture to receive the first pin 140 and a second hole 605 or aperture to receive the second pin 135.

The medical device 100 can include one or more additional or alternative components to facilitate coupling the first arm 115 with the second arm 120. For example, as depicted in at least FIG. 5, the medical device 100 (e.g., the pin 140) can include one or more retaining rings 305. The pin 140, or another component of the medical device 100, can include at least one shaft 310 to protrude through the first hole(s) 515. The shaft 310 can facilitate supporting the first arm 115 and the second arm 120 such that the first arm 115 can rotate or pivot relative to the second arm 120 by the shaft 310. The one or more retaining rings 305 can facilitate maintaining the shaft 310 or arms 115, 120 in position relative to one another in at least one direction. The pin 140, or another component of the medical device 100, can include at least one cap 535 that can facilitate securing the shaft 310 and retaining ring 305 in position. For example, the cap 535 can entirely cover the shaft 310 or the retaining ring 305. The retaining rings 305, shaft 310, or the caps 535 (e.g., the pin 140) can facilitate simplifying manufacturing of the first arm 115 with the second arm 120. For example, the connection simplifies manufacturing as compared to, for example, using screws, threads, or rivets.

The first tooth 145 can couple with the first arm 115 and the second tooth 150 can couple with the second arm 120. For example, as depicted in at least FIG. 5, the first tooth 145 or the second tooth 150 can include a protrusion 505 (e.g., a peg) extending from the tooth 145, 150. The protrusion 505 can facilitate coupling the tooth 145, 150 with a respective arm 115, 120. For example, the first arm 115 or the second arm 120 can include at least one aperture 510 to receive the protrusion 505 of the tooth 145, 150. The aperture 510 can be monolithically formed with the respective arm 115, 120 (e.g., via molding, as described herein). The protrusion 505 can include various features or mechanisms to facilitate coupling or fixing the protrusion 505 with the aperture 510. For example, the protrusion 505 can include one or more threads, flats, steps, ridges, ratchets (e.g., ratcheting mechanism), or other features. The aperture 510 can include various features to facilitate coupling the protrusion 505 with the aperture 510. For example, the aperture can include one or more threads, pawls, ratchets (e.g., ratcheting mechanism), or other features. The medical device 100 can include various additional or alternative components to facilitate coupling the protrusion 505 with the aperture 510. For example, the medical device 100 can include one or more adhesives or other components to facilitate coupling the protrusion 505 with the aperture 510.

The first tooth 145 and the second tooth 150 can be formed by various processes. For example, the first tooth 145 or the second tooth 150 can be formed by metal forging. This process can facilitate enhancing the accuracy/precision, strength, and resistance to fatigue and fracture of the teeth 145, 150. The first tooth 145 and the second tooth 150 can be formed of various types of metals including, but not limited to, a steel material (e.g., stainless steel). The first tooth 145 or the second tooth 150 can be formed through various additional or alternative processes including, but not limited to, rolling, extrusion, drawing, or stamping.

The first tooth 145 can couple with the first arm 115 by various processes. For example, the first tooth 145 can couple with the first arm 115 by press fitting the first tooth 145 with the molded first arm 115 (e.g., press fitting the protrusion 505 into the aperture 510). As another example, the first tooth 145 can couple with the first arm 115 by molding (e.g., overmolding) the first arm 115 around a portion of the first tooth 145 (e.g., around the protrusion 505 to form the aperture 510 around the protrusion 505). These processes can facilitate ensuring the connection between the first arm 115 and the first tooth 145 is secure and strong.

The second tooth 150 can couple with the second arm 120 by various processes. For example, the second tooth 150 can couple with the second arm 120 by press fitting the second tooth 150 with the molded second arm 120 (e.g., press fitting the protrusion 505 into the aperture 510). As another example, the second tooth 150 can couple with the second arm 120 by molding (e.g., overmolding) the second arm 120 around a portion of the second tooth 150 (e.g., the protrusion 505). These processes can facilitate ensuring the connection between the second arm 120 and the second tooth 150 is secure and strong. These manufacturing processes (e.g., molding the arms and flexors are unitary structures and coupling the teeth to the arm) can facilitate improving precision of manufacturing and strength of the teeth 145, 150 (e.g., via forging) while simplifying manufacturing of the arms 115, 120 and flexors 125, 130 (e.g., via molding).

FIG. 6 depicts an example sectional view of the medical device 100 cut along line 6-6 in FIG. 4. FIG. 7 depicts another example sectional view of the medical device 100 cut along line 7-7 in FIG. 1. The medical device 100 can cut various types of bones, tissue, or other body parts. For example, the medical device 100 can cut back bones or tissue at or near various digits of the hands or feet (e.g., fingers, toes). The first tooth 145 and the second tooth 150 can facilitate cutting the bones or tissues. For example, the first tooth 145 or the second tooth 150 can include a cutting edge CE_{T} of an angled or rounded cutting surface 205 that facilitates cutting tissue or bone at the digits of the body. For example, as depicted at least throughout FIGS. 6 and 7, the cutting edge CE_{T} of the first tooth 145 or second tooth 150 can be in a range of 0.01 to 0.20 mm. For example, the cutting edge CE_{T} of the first tooth 145 or second tooth 150 can be in a range of 0.01 mm to 0.17 mm. For example, the cutting edge CE_{T} of the first tooth 145 or second tooth 150 can be in a range of 0.10 mm to 0.17 mm. For example, the cutting edge CE_{T} of the first tooth 145 or second tooth 150 can be in a range of 0.14 mm to 0.16 mm, or about 0.15 mm. For example, the cutting edge CE_{T} of the first tooth 145 or second tooth 150 can be about 0.1524 mm ± 0.0254 mm (e.g., 0.006 ± 0.001 inches). This sharp cutting edge CE_{T} can facilitate providing a sharp tip of the teeth 145, 150 to allow the teeth 145, 150 to cut small or tough areas of bone or tissue. For example, the cutting edge CE_{T} can facilitate providing a sharp cutting surface as opposed to, for example, a dull crushing surface (e.g., having a cutting edge greater than or equal to 17 mm). This sharp cutting edge CE_{T} can allow for using less force to cut through tissue or bone as compared to other cutting edges, which can facilitate promoting faster healing for patients. In some implementations, the cutting edge CE_{T} may be substantially larger or smaller than described herein (e.g., in a range of 0 mm to 0.10 mm, in a range of 0.2 mm to 1 mm).

As depicted in at least FIG. 5, the medical device 100 can include various features to facilitate enhancing performance (e.g., durability, usability, strength) of the medical device 100. For example, the first arm 115 or the second arm 120 can include one or more grips 520 disposed on an outer surface 540 of the arms 115, 120 to facilitate a user gripping the arms 115, 120. The grips 520 can be or can include one or more protrusions, steps, irregularities, grooves, or other features to facilitate interrupting a surface of the arms 115, 120. The one or more grips 520 can be formed via molding (e.g., the grips 520 can be integrally coupled with the arms 115, 120 via molding). The first arm 115 or the second arm 120 can include at least one groove 525 that can form a rib of the arms 115, 120 to facilitate the strength of the arms 115, 120 and reduce undesired bending of the arms 115, 120. The first flexor 125 or the second flexor 130 can include at least one protrusion (e.g., protruding surface) or groove 530 formed on a surface of the flexor 130, 125. For example, the flexors 130, 125 can include at least one surface protrusion or cutout to facilitate the strength of the flexors 130, 125 and reduce undesired bending of the flexors 130, 125.

The medical device 100 can vary in size. For example, the entire medical device 100 can extend a length L and a width W (FIG. 1). For example, the length L of the medical device 100 can be in a range of 100 mm to 200 mm. For example, the length L of the medical device 100 can be in a range of 120 mm to 145 mm. For example, the length L of the medical device 100 can be about 135 ± 25 mm (e.g., about 5.35 ± 1.00 inches). This length L can facilitate precision and comfortability in cutting back tissue, bones, or other components of digits of the body (e.g., as compared to larger bones or body parts). In some implementations, the length L of the medical device 100 can be substantially larger or smaller (e.g., in a range of 5 mm to 100 mm, in a range of 200 mm to 650 mm). When the medical device 100 is in an open position (e.g., when the flexors 125, 130 bias the arms 115, 120 apart, as depicted in at least FIG. 1), the width W of the medical device 100 can be in a range of 45 mm to 150 mm. For example, the width W of the medical device 100 can be in a range of 75 mm to 100 mm. For example, the width W of the medical device 100 can be about 85.6 ± 12.7 mm (e.g., about 3.37 ± 0.50 inches). This width W can facilitate precision and comfortability in cutting back tissue, bones, or other components of digits of the body (e.g., as compared to larger bones or body parts). In some implementations, the width W of the medical device 100 can be substantially larger or smaller (e.g., in a range of 5 mm to 45 mm, in a range of 150 mm to 500 mm). The width W_{T} of the teeth 145, 150 (FIG. 2) can be in a range of 0.5 mm to 10 mm. For example, the width W_{T} of the teeth 145, 150 can be in a range of 2 mm to 5 mm. For example, the width W_{T} of the teeth 145, 150 can be about 3.5 ± 0.5 mm (e.g., 0.138 ± 0.02 inches). This width W_{T} of the teeth 145, 150 can facilitate precision and comfortability in cutting back tissue, bones, or other components of digits of the body (e.g., as compared to larger bones or body parts). In some implementations, the teeth width W_{T} may be substantially larger or smaller (e.g., in a range of 0.1 mm to 0.5 mm, in a range of 10 mm to 50 mm).

The medical device 100 can be a single use device. For example, the medical device 100 (e.g., the first arm 115, the second arm 120, the first flexor 125, and the second flexor 130) can be at least partially formed of bio-based plastic, polymer, or carbon materials (e.g., polyactic acid, polyhydroxyalkanoate, starch-based plastics, bio-polyethylene, bio-polypropylene, or various other types of bio-based materials). For example, the first arm 115, the second arm 120, the first flexor 125, and the second flexor 130 can be formed of molding (e.g., injection molding) to facilitate high-volume manufacturing of the medical device 100. The medical device 100 can be disposed of after being used for a single patient. The bio-based plastic material of the medical device 100 can facilitate easy decomposition of the medical device 100. This single use can facilitate reducing or eliminating the need for central sterile processing (CSP) for decontamination between uses and reducing or eliminating lack of access to a device. The entire medical device 100 (e.g., the teeth 145, 150, the arms 115, 120, the flexors 125, 130) can be single use or a portion of the medical device 100 (e.g., the arms 115, 120, the flexors 125, 130) can be single use.

In use, a user can cut back tissue or bone at or near a digit of the hands of feet using the medical device 100. For example, a user can grip the first arm 115 and the second arm 120 (e.g., within the palm of a user’s hand). A user can apply a force to the first arm 115 and the second arm 120 to cause the first arm 115 and the second arm 120 to move towards one another. This force can cause the first tooth 145 to move towards the second tooth 150 such that the teeth 145, 150 can engage one another (e.g., contact) at the end most section of the teeth 145, 150 (e.g., at the cutting edge CE_{T}) such that the teeth 145, 150 can apply a cutting force at the cutting edge CE_{T} of the teeth 145, 150 (e.g., at a closed position of the medical device 100). The movement of the first arm 115 and the second arm 120 towards each other can cause the first flexor 125 and the second flexor 130 to apply opposing forces to cause the flexors 125, 130 to bias the first arm 115 and the second arm 120 back to the open position (e.g., when the teeth 145, 150 do not contact) when the user stops applying a force to the arms 115, 120.

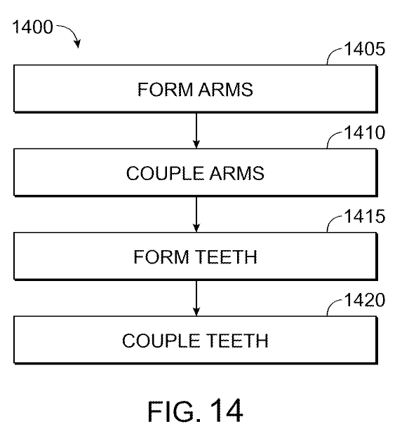

FIG. 14 depicts an example method 1400 of assembling the rongeur medical device 100, according to an example implementation. The method 1400 can include forming the one or more arms 115, 120 of the medical device 100, as depicted in act 1405. For example, the method 1400 can include forming, via molding (e.g., injection molding, overmolding, or another type of molding), the first arm 115 of the medical device 100. Molding the first arm 115 can include simultaneously molding the first arm 115 with the first flexor 125 and the first aperture 510 (e.g., such that the first arm 115, the first flexor 125, and the first aperture 510 form one unitary or monolithic molded structure). The method 1400 can include forming, via molding (e.g., injection molding, overmolding, or another type of molding), the second arm 120 of the medical device 100. Molding the second arm 120 can include simultaneously molding the second arm 120 with the second flexor 130 and the second aperture 510 (e.g., such that the second arm 120, the second flexor 130, and the second aperture 510 form one unitary or monolithic molded structure).

The method 1400 can include coupling the one or more arms 115, 120 together, as depicted in act 1410. For example, the method 1400 can include coupling the first arm 115 with the second arm 120 and the first flexor 125 with the second flexor 130. Coupling the first arm 115 with the second arm 120 can include overlapping at least a portion of the first arm 115 and the second arm 120 such that one or more holes 515 of the arms 115, 120 are aligned. Coupling the first arm 115 with the second arm 120 can include receiving, by the one or more holes 515, the shaft 310 such that the first arm 115 and the second arm 120 can rotatably couple by the shaft 310. Coupling the first arm 115 with the second arm 120 can include securing the shaft 310 with one or more retaining rings 305 or one or more caps 535. Coupling the first flexor 125 with the second flexor 130 can include coupling a pin 135 with one or more holes 605 of the flexors 125, 130 to couple the first flexor 125 with the second flexor 130 by the pin 135.

The method 1400 can include forming one or more teeth 145, 150, as depicted in act 1415. For example, the method 1400 can include forming, via forging (e.g., metal forging), the first tooth 145. The method 1400 can include forming, via forging (e.g., metal forging), the second tooth 150. The method 1400 can include forming the one or more teeth 145, 150 through various other means including, but not limited to, drawing, stamping, rolling, or extrusion.

The method 1400 can include coupling the one or more teeth 145, 150 with the one or more arms 115, 120, as depicted in act 1420. For example, the method 1400 can include coupling the first tooth 145 with the first arm 115 at the first aperture 510. The method 1400 can include coupling the second tooth 150 with the second arm 120 at the second aperture 510. Coupling the first tooth 145 with the first arm 115 at the first aperture 510 can include press fitting the first tooth 145 into the first aperture 510. Coupling the first tooth 145 with the first arm 115 at the first aperture 510 can include overmolding the first arm 115 around a portion of the first tooth 145 (e.g., around the protrusion 505 of the tooth 145). Coupling the second tooth 150 with the second arm 120 at the second aperture 510 can include press fitting the second tooth 150 into the second aperture 510. Coupling the second tooth 150 with the second arm 120 at the second aperture 510 can include overmolding the second arm 120 around a portion of the second tooth 150 (e.g., around the protrusion 505 of the tooth 150).

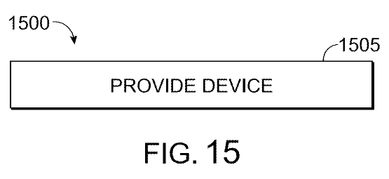

FIG. 15 depicts an example method 1500, according to an example implementation. The method 1500 can include providing the medical device 100, as depicted in act 1505. As described herein, the medical device 100 can include the first molded arm 115 having the first aperture 510 and the first flexor 125 monolithically formed with the first arm 115. The medical device 100 can include the second molded arm 120 coupled with the first arm 115. The second arm 120 can include the second aperture 510 and the second flexor 130 monolithically formed with the second arm 120. The medical device 100 can include the first tooth 145 coupled with the first arm 115 at the first aperture 510 (e.g., via press fitting or overmolding). The medical device 100 can include the second tooth 150 coupled with the second arm 120 at the second aperture 510 (e.g., via press fitting or overmolding). At least one of the first tooth 145 or the second tooth 150 can include a cutting edge CE_{T} being in a range of 0.10 to 0.17 mm (e.g., about 15 mm).

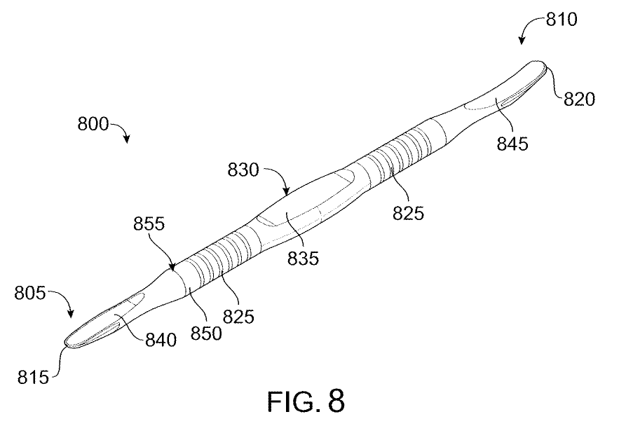

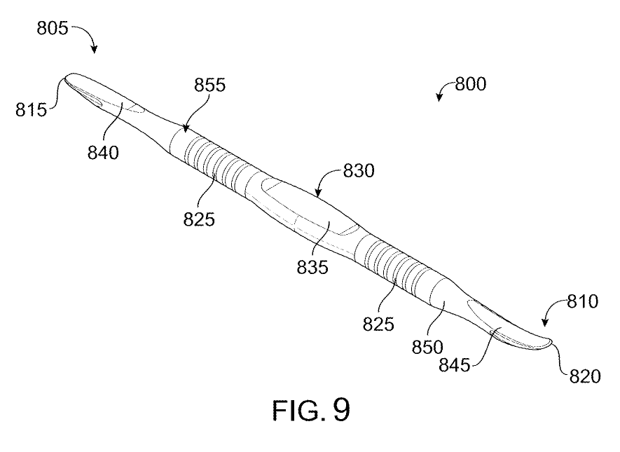

FIG. 8 and 9 depict example perspective views of a device (e.g., a medical device) 800, according to an example implementation. The medical device 800 can be used in one or more medical settings. For example, the medical device 800 can facilitate healing a patient, the medical device 800 can facilitate one or more medical professionals during operations or other activities, or the medical device 800 can be used in various other applications. For example, the medical device 800 can be used for a patient undergoing various procedures such as, for example, dissection, tissue manipulation, and separating structures of various digits of the hand or feet, or various other procedures. As described herein, the medical device 800 can be used to elevate, separate, or manipulate bone, tissue, or other components (e.g., nails) during a medical procedure. The medical device 800 can include or can be, a freer elevator medical device.

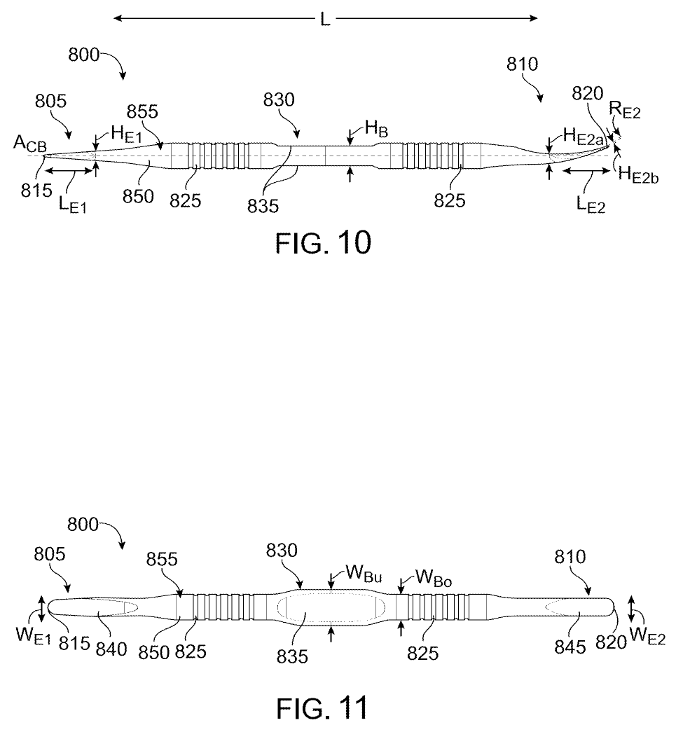

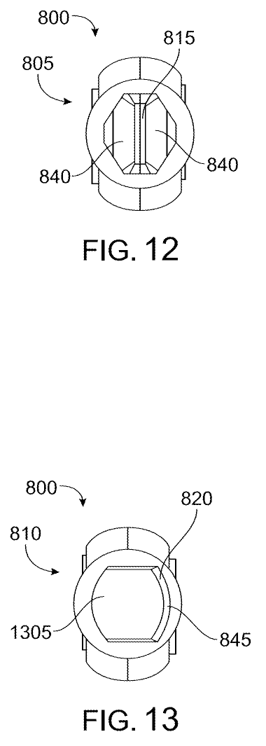

FIG. 10 depicts an example side view the medical device 800 and FIG. 11 depicts an example top view of the medical device 800. As depicted in at least FIGS. 8-11, the medical device 800 can include a body 855. The body 855 can extend between a first end 805 and a second end 810 of the medical device 800. FIG. 12 depicts an example front view of the medical device 800 and FIG. 13 depicts an example rear view of the medical device 800. As depicted in at least FIGS. 8-13, the first end 805 of the medical device 800 can oppose the second end 810 of the medical device 800. The first end 805 can be positioned in other various positions relative to the second end 810 (e.g., adjacent to). The first end 805 can be or can include a straight, wedged end. The second end 810 can be or can include a curved end. For example, the medical device 800 can be at least partially asymmetrical. The first end 805 (e.g., the straight end) can include a tip 815 defined by two substantially flat surfaces 840 extending from the body 855 towards the first end 805 and converging at the tip 815 to form a wedge shape. For example, the first end 805 can include a wedge shape that tapers in height (e.g., thickness) from the body 855 towards the tip 815. For example, the first end 805 can decrease in thickness from a proximal region towards the distal region of the first end 805. The tip 815 can include a point, or the tip 815 can include an edge that extends along a portion of the surface 840. The tip 815 can extend substantially straight outward from the body 855. For example, the tip 815 can be substantially aligned with the center axis A_{CB} (FIG. 10) of the body 855, such that the center axis A_{CB} of the body 855 extends through the tip 815 (e.g., such that the surfaces 840 converge to the center axis A_{CB} of the body 855). For example, the opposing surfaces 840 can be substantially symmetrical about the center axis A_{CB} of the body 855, as depicted in at least FIG. 10. The first end 805 can be used, for example, for elevating or manipulating periosteum of a digit of the hands or feet (e.g., tissue that surrounds bone, such as a finger or toe bone).

The second end 810 of the medical device 800 can include a tip 820 defined by at least one curved surface 845 extending from the body 855 towards the second end 810. The tip 820 can include a point, or the tip 820 can include an edge that extends along a portion of the surface 845. The second end 810 can be used, for example, for elevating, lifting, or manipulating a nail (e.g., such as a fingernail or toenail). As depicted in at least FIG. 13, the second end 810 of the medical device 800 can be defined by a first curved surface 845 and a second opposing curved surface 1305 that converge to the tip 820 of the second end 810. For example, the second end 810 can include a curved shape that tapers in height (e.g., thickness) from the body 855 to the tip 820. For example, the second end 810 can decrease in thickness from a proximal region towards the distal region of the second end 810. The tip 820 can curve such that the tip 820 is offset from a center axis A_{CB} of the body 855 of the medical device 100. For example, the second end 810 of the medical device 800 can include a curved shape that curves outward away from the center axis A_{CB} of the body 855 to define a radius of curvature, as described herein.

The medical device 800 can include one or more features that facilitate usability of the device 800. For example, the medical device 800 can include one or more grips 825 disposed on the outer surface 850 of the medical device 800 (e.g., outer surface 850 of the body 855). For example, as depicted in at least FIGS. 10 and 11, the grips 825 can include alternating depths of the outer surface 850 that form one or more grooves along the outer surface 850 such that the outer surface 850 of the body 855 is interrupted (e.g., ribbed). The grips 825 can be or include alternative or additional features including, but not limited to, protrusions, rubber grips, or other features to facilitate gripping the medical device 800. The grips 825 can be disposed at a portion of the medical device 800 that is substantially cylindrical in shape (e.g., at the body 855) to facilitate gripping the medical device 800.

The medical device 800 can include a bulge 830. The bulge 830 can couple to the body 855. For example, the bulge 830 can monolithically form with the body 855 (e.g., the body 855 and the bulge 830 can be formed via molding, or through various other processes). The bulge 830 can be disposed substantially near a center portion of the medical device 800 (e.g., in an axial direction) between the first end 805 and the second end 810 of the medical device 800. As depicted in at least FIG. 11, the bulge 830 can include a width W_{Bu} that extends outward beyond the width of other portions of the medical device (e.g., the bulge width W_{Bu} can be greater than the largest width of the body W_{Bo}). For example, the bulge 830 can include two substantially flat and opposing surfaces 835 that form the bulge 830 to facilitate preventing or reducing the medical device 800 from rolling along a surface (e.g., a medical tray or other substantially flat surface). In other words, the bulge 830 can be formed of a different shape from other portions of the medical device 800 (e.g., cylindrical) such that at least a portion of the bulge 830 engages with a surface (e.g., medical tray) if the medical device 800 begins to roll on the surface to prevent or reduce rolling of the medical device 800. The bulge 830 can additionally or alternatively facilitate gripping the medical device 800. For example, the opposing flat surfaces 835 can facilitate forming a section of the device 800 for a user to grip.

The body 855 can be substantially cylindrical in shape. For example, at least a portion of the body 855 (e.g., the portions of the body 855 adjacent to the bulge 830 having one or more grips 825) can be cylindrical in shape. One or more portions of the body 855 can include a tapering cylindrical shape. For example, the body 855 can taper towards the first end 805 and towards the second end 810 of the medical device 800. The bulge 830 can differ in shape from at least a portion of the body 855. For example, the bulge 830 can include a flattened, oblong cylindrical shape (e.g., having a lesser height than the body 855, but a greater width than the body 855, as described herein). The bulge 830 can include additional or alternative shapes including, but not limited to, cylindrical, elliptical cylindrical shape, obround shape, capsule shape, lozenge-shaped prism, rectangular, rounded rectangular, or various other shapes.

As depicted in at least FIG. 10, the first end 805 of the medical device 800 can taper between the portion of the medical device 800 having grips 825 (e.g., the body 855) to the tip 815 of the first end 805. The tapering of the opposing flat surfaces 840 of the first end 805 can facilitate providing a stiff tip 815 at the first end 805. For example, the first end 805 can have a higher rigidity or stiffness than the second end 810 of the medical device 800. For example, as described herein, a height (e.g., thickness) of the first tip 815 can be greater than a height (e.g., thickness) of the second tip 820 at least at one portion of the tips 815, 820. The tapered first end 805 can vary in size. For example, a length L_{E1} of the first end 805 at, for example, where the tip 815 couples with the body 855 to the endmost portion of the tip 815, can be in a range of 5 mm to 25 mm. For example, the length L_{E1} of the first end 805 at the tip 815 can be in a range of 10 mm to 15 mm. For example, the length L_{E1} of the first end 805 at the tip 815 can be about 13.7 mm (e.g., about 0.54 inches). This length L_{E1} can facilitate providing enough stiffness for, for example, manipulating or elevating periosteum while reducing or minimizing undesired manipulation of other tissue. This can facilitate promoting healing of a patient. This length may be about, for example, a quarter to a half of the distance of the tapering section of the first end 805 of the medical device 800. In other words, the length of the entirety of the first end 805 of the medical device 800 (e.g., the tapering section of the medical device 800 at the first end 805) can be in a range of 10 mm to 100 mm, or about 35-50 mm. In some implementations, the length L_{E1} can be substantially larger or smaller (e.g., in a range of 0 to 5 mm, in a range of 25 to 500 mm).

The height H_{E1} of the first end 805 at, for example, where the tip 815 couples with the body 855, can be in a range of 0.5 mm to 10 mm. For example, the height H_{E1} of the first end 805 at the tip 815 can be in a range of 1 mm to 5 mm, or in a range of 1 mm to 4 mm. For example, the height H_{E1} of the first end 805 at the tip 815 can be about 2.54 ± 0.76 mm (e.g., about 0.10 ± 0.03 inches). This height H_{E1} can facilitate providing enough stiffness to, for example, manipulating or elevating periosteum while reducing or minimizing undesired manipulation of other tissue. This height H_{E1} may be the height at, for example, about halfway (e.g., ¼ - ½) into the tapering section of the first end 805 of the medical device 800. In some implementations, the height H_{E1} can be substantially larger or smaller (e.g., in a range of 0 to 1 mm, in a range of 5 to 100 mm).

As depicted in at least FIG. 11, a width W_{E1} of the first end 805 at the end most section of the first end 805 (e.g., the end most portion of the tip 815) can be in a range of 1 mm to 15 mm. For example, the width W_{E1} of the first end 805 can be in a range of 1 mm to 6 mm. For example, the width W_{E1} of the first end 805 can be about 4.10 mm ± 1.27 mm (e.g., about 0.16 ± 0.05 inches). This width W_{E1} can facilitate providing enough stiffness to, for example, manipulating or elevating periosteum while reducing or minimizing undesired manipulation of other tissue. In some implementations, the width W_{E1} can be substantially larger or smaller (e.g., in a range of 0 to 1 mm, in a range of 5 to 100 mm).

As depicted in at least FIG. 10, the second end 810 can taper in height (e.g., thickness) between the portion of the medical device 800 having grips 825 (e.g., the body 855) to the tip 820 of the second end 810. The tapering of the curved surfaces 845, 1305 of the second end 810 and the curvature of the second end 810 can facilitate lifting a nail (e.g., fingernail or toenail) gradually as the medical device 800 (the tip 820) is pushed, for example, between the nail and the finger or toe in connection with the nail. The tapered and curved second end 810 can vary in size. For example, a length L_{E2} of the second end 810 at the tip 820 (e.g., where the tip 820 couples with the body 855) can be in a range of 5 mm to 25 mm. For example, the length L_{E2} of the second end 810 at the tip 820 can be in a range of 10 mm to 15 mm. For example, the length L_{E2} of the second end 810 at the tip 820 (e.g., where the tip 820 couples with the body 855) can be about 14.7 mm (e.g., about 0.58 inches). This length may be about, for example, a quarter to a half of the distance of the tapering section of the second end 810 of the medical device 800. In other words, the length of the entirety of the second end 810 of the medical device 800 (e.g., the tapering section of the medical device 800 at the second end 810) can be in a range of 10 mm to 100 mm, or about 35-50 mm. In some implementations, the length L_{E2} can be substantially larger or smaller (e.g., in a range of 0 to 5 mm, in a range of 25 to 500 mm).

The height H_{E2a} of the second end 810 at the tip 820 can be in a range of 0.5 mm to 10 mm. For example, the height H_{E2a} of the second end 810 at the tip 820 can be in a range of 1 mm to 5 mm, or in a range of 1 mm to 4 mm. For example, the height H_{E2a} of the second end 810 at the tip 820 can be about 2.29 ± 0.76 mm (e.g., about 0.09 ± 0.03 inches). This height H_{E2a} may be the height at, for example, about halfway (e.g., ¼ - ½) into the tapering section of the second end 810 of the medical device 800. In some implementations, the height H_{E2a} can be substantially larger or smaller (e.g., in a range of 0 to 1 mm, in a range of 5 to 100 mm).

A height H_{E2b} of the second end 810 at the end most section of the tip 820 can be in a range of 0 mm to 3 mm. For example, the height H_{E2b} of the second end 810 at the end most portion of the tip 820 can be in a range of 0 mm to 1 mm. For example, the height H_{E2b} of the second end 810 at the end most section of the tip 820 can be about 0.51 mm ± 0.254 mm (e.g., about 0.02 ± 0.01 inches). In some implementations, the height H_{E2b} can be substantially larger or smaller (e.g., in a range of 3 to 75 mm).

A radius R_{E2} of curvature of the tip 820 at the second end 810 can be in a range of 20 mm to 75 mm. For example, the radius R_{E2} of curvature of the tip 820 at the second end 810 can be in a range of 30 mm to 60 mm. For example, the radius R_{E2} of curvature of the tip 820 at the second end 810 can be about 38.1 mm with a tolerance of + 19.05 mm / - 6.35 mm (e.g., about 1.50 inches with a tolerance of + 0.75 / - 0.25 inches). In some implementations, the radius R_{E2} of curvature of the tip 820 can be substantially larger or smaller (e.g., in a range of 0 to 20 mm, or in a range of 75 to 200 mm).

As depicted in at least FIG. 11, a width W_{E2} of the second end 810 at the tip 820 (e.g., at the end most portion of the tip 820) can be in a range of 1 mm to 15 mm. For example, the width W_{E2} of the second end 810 at the tip 820 can be in a range of 1 mm to 10 mm. For example, the width W_{E2} of the second end 810 at the tip 820 can be about 5.10 mm ± 1.27 mm (e.g., about 0.20 ± 0.05 inches). In some implementations, the width W_{E2} of the second end 810 at the tip 820 can be substantially larger or smaller (e.g., in a range of 0 to 1 mm, or in a range of 15 to 100 mm).

The dimensions of the second end 810 (e.g., the length L_{E2}, height H_{E2a} at the start of the tip 820, height H_{E2b} at the end most portion of the tip 820, and the radius R_{E2} of curvature of the second end 810) can facilitate optimizing the second end 810 of the medical device 800 for gradually lifting a nail of a finger or toe as the tip 820 of the second end 810 is pushed between the nail and the finger. For example, the tip 820 can be long, thick, and angled (e.g., steep) enough to apply enough force to lift a nail, but can be thin, angled, and small enough to reduce undesired forces at a sensitive area of the nail. For example, the tip 820 can facilitate gradually elevating a nail of a digit (e.g., including a pinky toe or finger) while reducing or eliminating undesirable forces on the nail that could occur if, for example, the tip 820 were larger in size (e.g., greater than or equal to, for example, 4 mm).

As depicted in at least FIG. 11, the width W_{Bu} of the bulge 830 can be greater than the largest width W_{Bo} of the body 855 (e.g., the width of the outer surface 850). For example, the width W_{Bu} of the bulge 830 can be in a range of 1 mm to 20 mm. For example, the width W_{Bu} of the bulge 830 can be in a range of 5 mm to 15 mm. For example, the width W_{Bu} of the bulge 830 can be about 9.9 mm with a tolerance of + 2.54 mm (e.g., about 0.39 inches with a tolerance of + 0.10 inches). In some implementations, the width W_{Bu} of the body 855 can be substantially larger or smaller (e.g., in a range of 0 to 1 mm, or in a range of 20 to 100 mm).

The W_{Bo} of the body 855 can be in a range of 1 mm to 20 mm. For example, the width W_{Bo} of the body 855 can be in a range of 5 mm to 10 mm. For example, the width W_{Bo} of the body 855 can be about 7.11 mm ± 1.27 mm (e.g., about 0.28 ± 0.05 inches). As described herein, the body 855 can be substantially cylindrical in shape, such that the width W_{Bo} of the body 855 can be the same or substantially the same as a height of the body (e.g., the width W_{Bo} of the body 855 and the height of the body 855 can be defined be the diameter of the body 855). In some implementations, the width W_{Bo} of the body 855 can be substantially larger or smaller (e.g., in a range of 0 to 1 mm, or in a range of 20 to 100 mm).

The height H_{B} of the bulge 830 can be less than the height/width W_{Bo} of the body 855. For example, the height H_{B} of the bulge 830 can be in a range of 1 mm to 15 mm. For example, the height H_{B} of the bulge 830 can be in a range of 1 mm to 10 mm. For example, the height H_{B} of the bulge 830 can be about 5.28 mm with a tolerance of - 1.27 mm (e.g., about 0.208 inches with a tolerance of – 0.050 inches). In some implementations, the height H_{B} of the bulge 830 can be substantially larger or smaller (e.g., in a range of 0 to 1 mm, or in a range of 15 to 100 mm).

The medical device 800 can vary in size. For example, the medical device 800 can be sized or shaped for use for manipulating various digits of the hands or feet. For example, the length L of the medical device 800 can be in a range of 100 to 200 mm. For example, the length L of the medical device 800 can be in a range of 125 to 175 mm. For example, the length L of the medical device 800 can be about 152.4 mm ± 25.4 mm (e.g., about 6.00 inches ± 1.00 inches). In some implementations, the length L of the medical device 800 can be substantially larger or smaller (e.g., in a range of 0 to 100 mm, or in a range of 200 to 500 mm).

The medical device 800 can be a single use device. For example, the medical device 800 (e.g., the entirety of the medical device 800 or at least a portion of the medical device 800) can be at least partially formed of bio-based plastic, polymer, or carbon materials (e.g., polyactic acid, polyhydroxyalkanoate, starch-based plastics, bio-polyethylene, bio-polypropylene, or various other types of bio-based materials). The medical device 800 can be disposed of after being used for a single patient. The bio-based plastic material of the medical device 800 can facilitate easy decomposition of the medical device 800. This single use facilitates reducing or eliminating the need for central sterile processing (CSP) for decontamination between uses and reducing or eliminating lack of access to a device.

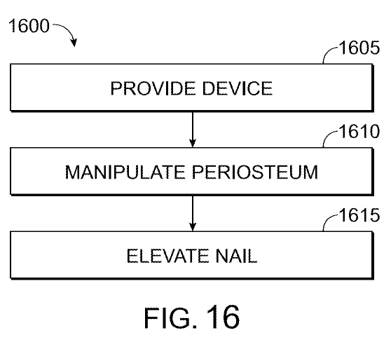

FIG. 16 depicts an example method 1600 of using the freer elevator medical device 800, according to an example implementation. The method 1600 can include providing the medical device 800, as depicted in act 1605. As described herein, the medical device 800 can include the body 855. The body 855 can extend between the first end 805 and the second end 810 of the medical device 800. The first end 805 of the medical device 800 can include a wedge shape that tapers in height from the body 855 toward the first tip 815. The first tip 815 can be substantially aligned with a center axis A_{CB} of the body 855 to facilitate providing a stiff end of the device 800. The second end 810 can include a curved shape that tapers in height/thickness from the body to the second tip 820. The second tip 820 can curve outward away from the center axis A_{CB} of the body 855 to define a radius of curvature R_{E2} in a range of, for example, 30 mm to 60 mm.

The method 1600 can include manipulating periosteum, as depicted in act 1610. For example, the method 1600 can include manipulating, by the tip 815 of the first end 805 of the freer elevator medical device 800, periosteum of a digit of a hand or foot (e.g., to push back the tissue, scrape bone to free bone from tissue). The method 1600 can include elevating a nail, as depicted in act 1615. For example, the method 1600 can include elevating, by the tip 820 of the second end 810 of the freer elevator medical device 800, a nail of the digit. With the size, shape, and configuration of the medical device 800 described herein, the device 800 can effectively manipulate or elevate portions of tissues or nails of a digit (e.g., finger or toe) of a body while reducing or minimizing undesirable pushing, pulling, or other forces on the digit. For example, as described herein, the device 800 can gently and gradually push back periosteum using the first end 805 or elevate a nail using the second end 810 while facilitating promoting a patient’s healing process post-operation. For example, using the first end 805 to scrape away tissue as opposed to, for example, cutting away the tissue can facilitate reducing cutting off blood supply so more blood can be delivered to the digit to promote healing, providing a cleaner and higher quality procedure).

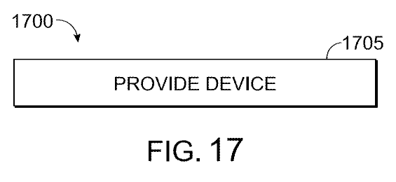

FIG. 17 depicts a method 1700, according to an example implementation. The method 1700 can include providing the medical device 800, as depicted in act 1705. As described herein, the medical device 800 can include the body 855. The body 855 can extend between the first end 805 and the second end 810 of the medical device 100. The medical device 800 can include the bulge 830 coupled with the body 855. The bulge 830 can be disposed between the first end 805 and the second end 810 of the medical device 800. The width W_{Bu} of the bulge 830 can be greater than a width W_{Bo} of the body 855. The first end 805 of the medical device 800 can include a wedge shape that tapers in height from the body 855 toward the first tip 815. The first tip 815 can be substantially aligned with a center axis A_{CB} of the body 855 to facilitate providing a stiff end of the device 800. The second end 810 can include a curved shape that tapers in height from the body to the second tip 820. The second tip 820 can curve outward away from the center axis A_{CB} of the body 855 to define a radius of curvature R_{E2} in a range of 30 mm to 60 mm. A height H_{E2b} at an end most portion of the tip 820 can be in a range of 0 mm to 1 mm. This configuration can facilitate gently and gradually lifting a nail.

While operations are depicted in the drawings in a particular order, such operations are not required to be performed in the particular order shown or in sequential order, and all illustrated operations are not required to be performed. Actions described herein can be performed in a different order.

Having now described some illustrative implementations, it is apparent that the foregoing is illustrative and not limiting, having been presented by way of example. In particular, although many of the examples presented herein involve specific combinations of method acts or system elements, those acts and those elements may be combined in other ways to accomplish the same objectives. Acts, elements and features discussed in connection with one implementation are not intended to be excluded from a similar role in other implementations.

The phraseology and terminology used herein is for the purpose of description and should not be regarded as limiting. The use of “including” “comprising” “having” “containing” “involving” “characterized by” “characterized in that” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

Any references to implementations or elements or acts of the systems and methods herein referred to in the singular may also embrace implementations including a plurality of these elements, and any references in plural to any implementation or element or act herein may also embrace implementations including only a single element. References in the singular or plural form are not intended to limit the presently disclosed systems or methods, their components, acts, or elements to single or plural configurations. References to any act or element being based on any information, act or element may include implementations where the act or element is based at least in part on any information, act, or element.

Any implementation disclosed herein may be combined with any other implementation or embodiment, and references to “an implementation,” “some implementations,” “one implementation” or the like are not necessarily mutually exclusive and are intended to indicate that a particular feature, structure, or characteristic described in connection with the implementation may be included in at least one implementation or embodiment. Such terms as used herein are not necessarily all referring to the same implementation. Any implementation may be combined with any other implementation, inclusively or exclusively, in any manner consistent with the aspects and implementations disclosed herein.

References to “or” may be construed as inclusive so that any terms described using “or” may indicate any of a single, more than one, and all of the described terms. References to at least one of a conjunctive list of terms may be construed as an inclusive OR to indicate any of a single, more than one, and all of the described terms. For example, a reference to “at least one of ‘A’ and ‘B’” can include only ‘A’, only ‘B’, as well as both ‘A’ and ‘B’. Such references used in conjunction with “comprising” or other open terminology can include additional items.

Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any claim elements.

Modifications of described elements and acts such as variations in sizes, dimensions, structures, shapes and proportions of the various elements, values of parameters, mounting arrangements, use of materials, colors, orientations can occur without materially departing from the teachings and advantages of the subject matter disclosed herein. For example, elements shown as integrally formed can be constructed of multiple parts or elements, the position of elements can be reversed or otherwise varied, and the nature or number of discrete elements or positions can be altered or varied. Other substitutions, modifications, changes and omissions can also be made in the design, operating conditions and arrangement of the disclosed elements and operations without departing from the scope of the present disclosure.

Systems and methods described herein may be embodied in other specific forms without departing from the characteristics thereof. For example, the medical device 100 can be used with various components including, but not limited to, a femoral component, a pelvic component, a tibia component, a skeletal component, a humeral component, a shoulder component, or other various components. Further relative parallel, perpendicular, vertical or other positioning or orientation descriptions include variations within +/-10% or +/-10 degrees of pure vertical, parallel or perpendicular positioning. References to “approximately,” “about” “substantially” or other terms of degree include variations of +/-10% from the given measurement, unit, or range unless explicitly indicated otherwise. Coupled elements can be electrically, mechanically, or physically coupled with one another directly or with intervening elements. Scope of the systems and methods described herein is thus indicated by the appended claims, rather than the foregoing description, and changes that come within the meaning and range of equivalency of the claims are embraced therein.

What is claimed is:

1. A rongeur medical device, comprising:a first molded arm having a first aperture and a first flexor monolithically coupled with the first molded arm;a second molded arm coupled with the first molded arm, the second molded arm having a second aperture and a second flexor monolithically coupled with the second molded arm;a first tooth coupled with the first molded arm at the first aperture;a second tooth coupled with the second molded arm at the second aperture; and at least one of the first tooth or the second tooth having a cutting edge in a range of 0.10 mm to 0.17 mm.

2. The rongeur medical device of claim 1, comprising:the first molded arm coupled with the second molded arm by a first pin; andthe first flexor to couple with the second flexor by a second pin.

3. The rongeur medical device of claim 1, comprising:the first flexor to couple with the second flexor; andthe first flexor and the second flexor to bias the first molded arm and the second molded arm away from one another.

4. The rongeur medical device of claim 1, comprising:the first tooth including a protrusion;the first aperture to receive the protrusion to couple the first tooth with the first molded arm; and at least one of the protrusion or the first aperture including a ratcheting mechanism to fix the protrusion to the first aperture.

5. The rongeur medical device of claim 1, comprising:the first tooth including a protrusion; andthe first aperture to receive the protrusion to couple the first tooth with the first molded arm.

6. The rongeur medical device of claim 1, comprising:an outer surface of the first molded arm having an irregularity to form a grip; andthe irregularity of the outer surface integrally coupled with the first molded arm.

7. The rongeur medical device of claim 1, comprising:the first molded arm coupled to the second molded arm by a shaft and a retaining ring.

8. The rongeur medical device of claim 1, comprising:the first flexor of the first molded arm including at least one of a groove or a protruding surface.

9. The rongeur medical device of claim 1, comprising:the first molded arm including a groove to form a rib.

10. The rongeur medical device of claim 1, comprising:the first molded arm comprising a bio-based material.

11. The rongeur medical device of claim 1, comprising:the cutting edge in a range of 0.14 mm to 0.16 mm.

12. The rongeur medical device of claim 1, comprising:
the first tooth comprising a steel material.

13. The rongeur medical device of claim 1, comprising:a length of the rongeur medical device in a range of 120 mm to 145 mm.

14. The rongeur medical device of claim 1, comprising:a width of the rongeur medical device in a range of 75 mm to 100 mm in an open position of the rongeur medical device.

15. A method of assembling a rongeur medical device, comprising:forming, via molding, a first arm, the first arm including a first flexor and a first aperture;forming, via molding, a second arm, the second arm including a second flexor and a second aperture;coupling the first arm with the second arm and the first flexor with the second flexor;forming, via forging, a first tooth;forming, via forging, a second tooth;coupling the first tooth with the first arm at the first aperture; andcoupling the second tooth with the second arm at the second aperture.

16. The method of assembling the rongeur medical device of claim 15, comprising:coupling the first tooth with the first arm at the first aperture comprising press fitting a portion of the first tooth into the first aperture.

17. The method of assembling the rongeur medical device of claim 15, comprising:coupling the first tooth with the first arm at the first aperture comprising overmolding the first arm around a portion of the first tooth.

18. The method of assembling the rongeur medical device of claim 15, comprising:coupling the first arm with the second arm including receiving, by a first hole in the first arm and a second hole in the second arm, a shaft, and securing the shaft with at least one retaining ring.

19. A method, comprising:providing a rongeur medical device, including:a first molded arm having a first aperture and a first flexor monolithically coupled with the first molded arm;a second molded arm coupled with the first molded arm, the second molded arm having a second aperture and a second flexor monolithically coupled with the second molded arm;a first tooth coupled with the first molded arm at the first aperture;a second tooth coupled with the second molded arm at the second aperture; and at least one of the first tooth or the second tooth having a cutting edge in a range of 0.10 mm to 0.17 mm.

20. The method of claim 19, comprising:the cutting edge being in a range of 0.14 mm to 0.16 mm.

21-40. (canceled)
